(19) United States
(12) Patent Application Publication
(10) Pub. No.: US 2002/0055714 A1
(43) Pub. Date: May 9, 2002

(54) Method and apparatus for the non-surgical placement of a subcutaneously tunnelled catheter through the skin into a central vein

(76) Inventor: Richard B. Rothschild, Thousand Oaks, CA (US)

Correspondence Address: Jack C. Munro, Agent of Record, Suite 225, 28720 Roadside Drive, Agoura Hills, CA, 91301, US

(21) Appl. No.: 10/034,024

(22) Filed: Dec. 19, 2001

Related U.S. Application Data
(63) Continuation of application No. 09/313,608, filed on May 18, 1999.

(51) Int. Cl. A61M 5/178

(52) U.S. Cl. 604/164.1; 604/164.13

(57) ABSTRACT

The non-surgical method of installing a subcutaneously tunneled semipermanent catheter through the skin and into a central vein which utilizes an introducer catheter which has a pair of proximal ports, distal port, and an intermediate port located between the proximal ports and the distal port. The distal section of the introducer catheter is to be positioned within the central vein and the intermediate port is positioned exterior of but directly adjacent the vein and under the skin. A guide wire redirecting catheter is inserted into the introducer catheter and engages with the intermediate port. A sharp tipped guide wire is inserted through the guide wire redirecting catheter and is discharged from the redirecting catheter and travels subcutaneously some distance before penetrating the skin. The portion of the sharp tipped guide wire within the introducer catheter is then prolapsed into the distal portion of the introducer catheter. A dilator catheter is then inserted over the distal section of the sharp tipped guide wire and through the intermediate port into the distal section of the main introducer catheter. The sharp tipped guide wire is then replaced with an extra support guide wire which is inserted within this dilator catheter. The introducer catheter is then removed. The dilator catheter is then removed leaving the extra support guide wire in place which has coursed subcutaneously some distance before entering the central vein for access for the placement of a semipermanent catheter.

### REFERENCE TO PRIOR APPLICATION

This application is a continuation of patent application Ser. No. 09/313,608, filed May 18, 1999 by the same title and same inventor.

### BACKGROUND OF THE INVENTION

1) Field of the Invention

This invention allows for the placement of a subcutaneously tunneled intravenous catheter that can be placed percutaneously at any bedside and will allow for lower infection rates and longer temporal permanency the catheter.

2) Description of the Prior Art

Within hospitals, it is, at times, necessary to install a catheter into a vein of a patient on a semipermanent basis to be used for the administration of medicines, nutrition, fluids and for intravenous access to allow for monitoring for cardiac function, cardiac pacing or hemodialysis. The catheter may remain in place for an extended period of time, such as days to weeks and possibly even months. It has been observed that serious infections can be introduced by intravenous catheters and this risk increases with the duration that the intravenous catheter remains in place. These infections occur because the catheter punctures the skin at the closest access site to the vein, and bacteria present on the skin may migrate along the course of the catheter into the vein. This problem is aggravated if a catheter works itself outward, which is a normal occurrence, and is then readvanced into the vein. When this occurs, the proximal portion of the catheter becomes contaminated by the non-sterile skin and on readvancing the catheter, this contaminated portion of the catheter is advanced into the vein introducing bacteria to the bloodstream that can lead to serious infections.

In order to avoid this kind of infection, it has been common to utilize a subcutaneously tunneled catheter, such as a Groshan or Broviac catheter. Placement of these catheters requires a surgical procedure. A surgical procedure, defined by this invention, requires a patient to be moved to an operating room, the patient to be anesthetized and a medical doctor to make an incision some distance away from the vein and surgically tunnel the catheter subcutaneously some distance before the catheter enters the vein. This procedure, is expensive and involves a high degree of risk and discomfort to the patient. However, because the catheter punctures the skin at some distance from the vein, bloodstream infections are extremely rare.

There is a need to achieve a non-surgical procedure for the installing of a subcutaneously tunneled catheter so as to reduce costs and discomfort to the patient and allow for longer permanency of the catheter and to reduce the risk of any infection being introduced by the catheter into the bloodstream.

### SUMMARY OF THE INVENTION

One of the primary objectives of the present invention is to install a semipermanent catheter within a vein where the proximal end of the catheter punctures the skin some distance from the vein and tunnels subcutaneously some distance before it enters the vein so as to minimize the possibility of infection occurring within the blood.

Another objective of the present invention is to achieve semipermanent installation of a catheter within a vein without requiring a surgical procedure thereby decreasing risk and discomfort to the patient.

Another objective of the present invention is to achieve a semipermanent installation of a catheter within a vein which is substantially less costly than the current surgically placed catheter procedure.

Another objective of the present invention is to provide for a semipermanent installation of a catheter within a vein with such installation being achieved at the bedside and not requiring the use of a separate facility, such as an operating room.

A medical practitioner, such as a doctor, starts the non-surgical placement of the semipermanent catheter in the form of a subcutaneously tunneled intravenous catheter (STIC) by inserting an eighteen gauge thin walled needle into a central vein and then inserting a guide wire (GW) through the needle. The needle is then removed and an introducer catheter (IC) which contains a dilator catheter (DC) is then inserted over the GW into the central vein utilizing the standard modified Sellinger technique. This IC has a single lumen with a proximal end which has two proximal ports through one of which courses the GW and the DC. This DC, when in place, prevents blood from being aspirated from the distal port when the DC is in place within the IC. Located between the two proximal ports and the single distal port of this IC there is an intermediate port. The proximal portion of this IC that is defined as the part of the IC located between the proximal ports and the intermediate port is wider than the distal portion of the IC which is the part of the IC between the intermediate port and the distal port. Running between the intermediate port and the distal port there is located a frangible longitudinal seam. The intermediate port is then positioned directly adjacent but exterior to the vein with the distal port still within the central vein. A guide wire redirecting catheter (GWRC) is then advanced through the unoccupied proximal port of the IC to engage the intermediate port. A sharp tipped guide wire (STGW) is then advanced through the GWRC and is manipulated to tunnel subcutaneously some distance and then exit through the surface of the skin. The GWRC, the DC and the GW are then removed. The DC is placed over the proximal part of the STGW causing a portion of the proximal part of the STGW to prolapse into the distal portion of the IC. The DC is then removed. A second DC with a wider proximal portion is advanced over the distal part of the STGW and courses subcutaneously some distance and then through the intermediate port into the distal portion of the second DC. The STGW is then removed from the IC and into the lumen of the vein. An extra support guide wire (ESGW) is then inserted within the second DC into the distal portion of the IC and into the lumen of the vein. The IC is then removed by having the wider portion of the second DC forced through the distal portion of the IC causing breakage of the frangible, longitudinal seam. After the IC is removed, the second DC is then removed leaving the ESGW which travels subcutaneously some distance before it enters the vein. This ESGW is then used to gain access into the vein by any catheter or sheath desired.

### BRIEF DESCRIPTION OF THE DRAWINGS

FIG. 1 is an isometric view of the IC that is used in conjunction with this invention;

FIG. 2 is a cross-sectional view through the distal end of the IC taken along line 2-2 of FIG. 1 showing a longitudinal, frangible seam included within the distal end of the IC;

FIG. 3 is an isometric of a DC that is used in conjunction with this invention;

FIG. 3A is an isometric view of a GWRC that is utilized in conjunction with this invention;

FIG. 4 is a side view of a STGW that is utilized in conjunction with this invention;

FIG. 5 is an enlarged view taken along line 5-5 of FIG. 4 showing more clearly the boundary between a stiffer portion of the STGW and a more flexible portion of the STGW;

FIG. 6 is a diagrammatic view depicting inserting of a thin walled eighteen gauge needle into a vein with the proximal end of the needle passing through the skin at the closest access site to the vein;

FIG. 7 is a view similar to FIG. 6 but with a first GW being inserted through the needle into the vein;

FIG. 8 is a view similar to FIG. 7 but with the needle now removed leaving only the first GW in position;

FIG. 9 is a view similar to FIG. 8 but where the IC with the DC in one of the proximal ports has been placed over the GW with both the distal end and the intermediate port of the IC being located within the vein;

FIG. 10 is a view similar to FIG. 9 but where the IC is in the process of being withdrawn so that the intermediate port is now located exteriorly of the vein but still below the skin within the subcutaneous tissue;

FIG. 10A is similar to FIG. 10 but with the GWRC having been advanced through the unoccupied proximal port of the IC such that the distal port of the GWRC has engaged the intermediate port of the IC;

FIG. 11 is a view similar to FIG. 10A but showing the insertion of a STGW through the GWRC and then tunneling subcutaneously some distance within the tissue;

FIG. 12 is a view similar to FIG. 11 but with the DC and first GW and the GWRC all removed from the IC;

FIG. 13 is a view similar to FIG. 12 showing the prolapsing of the STGW by inserting of the original DC over the proximal part of the STGW causing it to prolapse into the distal end of the IC;

FIG. 13A is a view similar to FIG. 13 but with the original DC removed;

FIG. 13B is an isometric view of the second DC used in conjunction with the STGW;

FIG. 14 is a view similar to FIG. 13A with the second DC inserted over the distal part of the STGW into the distal portion of the IC;

FIG. 14A is a view similar to FIG. 14 but with the STGW now being withdrawn and an ESGW inserted through the second DC that courses through the intermediate port of the IC;

FIG. 15 is a view similar to FIG. 14 showing partial removal of the IC from the vein and breaking of the longitudinal frangible seam; and

FIG. 16 is a view similar to FIG. 15 but with the IC and the second DC being completely removed from the vein with a STIC installed in place of the second DC.

### DETAILED DESCRIPTION OF THE PREFERRED EMBODIMENT

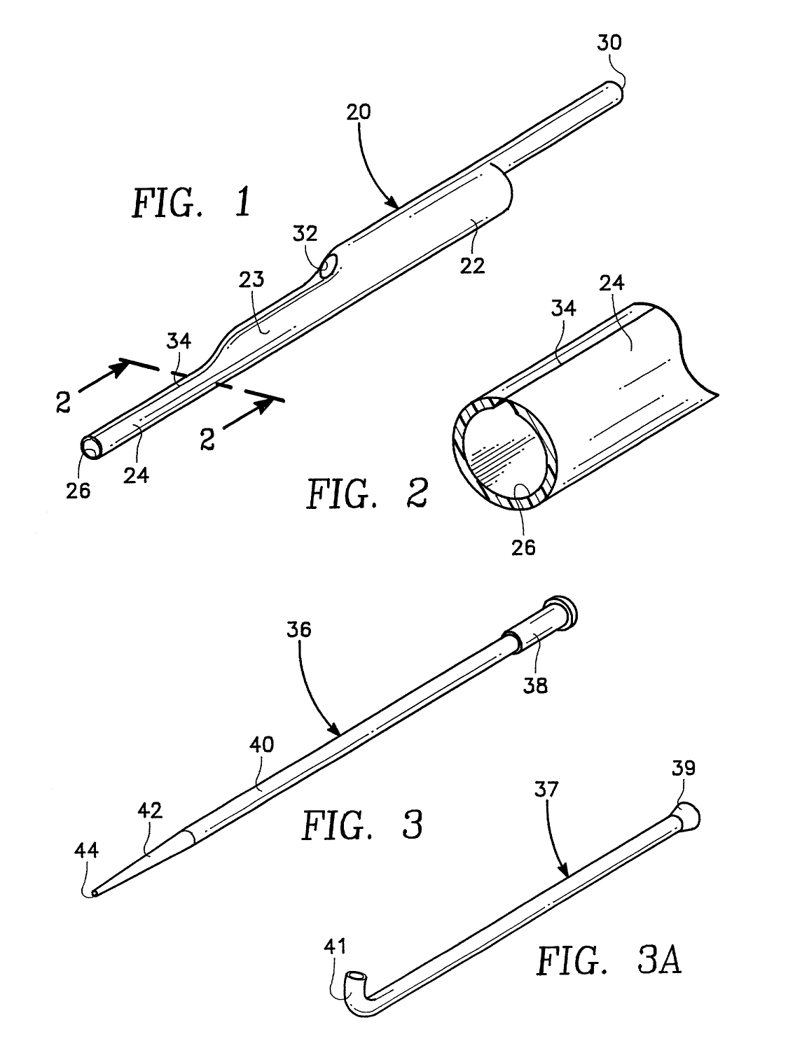

Referring particularly to FIGS. 1-9 of the drawings, there is shown the introducer catheter (IC) 20 that is utilized in conjunction with this invention. The IC 20 has a proximal portion 22, an intermediate portion 23 and a distal portion 24. The lumen of the proximal portion 22 is approximately 2.67 millimeters in diameter, the intermediate portion 23 is 2.33 millimeters in diameter and the distal portion 24 is 1.33 millimeters in diameter. The distal portion 24 terminates at the distal port 26 which also has a lumen of 1.33 millimeters. A common lumen 80 extends entirely through the IC 20. The common lumen 80 constitutes a single chamber resulting in the introducer catheter 20 being entirely hollow. Lumen 80 is not divided in any manner. At the proximal portion 22, the common lumen 80 terminates into a pair of proximal ports 28 and 30, which are in juxtaposition, with the proximal port 30 being longer than port 28 such that the length from proximal port 30 to an intermediate port 32 of the intermediate portion 23 is the same as the length of DC 36 which is described below. Both proximal ports 28 and 30 each are approximately 1.33 millimeters in diameter. The proximal ports 28 and 30 are separated by a thin wall 21. Between the proximal ports 28 and 30 and the distal port 26 is located the intermediate port 32 that connects to the common lumen of the IC 20. The intermediate port 32 is positioned at the dividing point between the proximal portion 22 and the intermediate portion 23. Along the distal portion 24 and also the intermediate portion 23 runs a longitudinal, frangible seam 34 which extends from the distal port 26 to the intermediate port 32. The function of this longitudinal, frangible seam 34 will be explained further on in the specification. The normal material of construction of the IC 20 will generally be plastic. The thickness of the IC 20 is sufficiently thin so that the IC 20 is bendable but yet will assume the at-rest configuration shown in FIG. 1.

Referring particularly to FIG. 3, there is shown the structure of a plastic DC 36 which has a proximal section 38 located at its proximal end and an elongated body 40, which measures 1.33 millimeters in diameter, extends from the proximal section 38 to a tip section 42 connected to the body 40 and located at the distal end of the DC 36. It is noted that the tip section 42 is tapered so that when a GW is passed through the distal port 44, the tip section 42 will readily slide into a small aperture within a vein because of the tapering of the tip section 42. It is also noted that the DC 36 intended to be inserted within proximal port 28 of the IC. When the DC 36 is within the IC 20 and located within a central vein (FIG. 9), blood can be aspirated from the proximal port 30 through intermediate port 32 but no blood can be aspirated from the distal port 26 because the DC forms a tight fit with the portion of the common lumen 80 that is located within the distal portion 24. Ports 28, 30 and lumen 43 of DC 36 are compatible with standard syringes.

Referring particularly to FIG. 3A, there is shown a guide wire redirecting catheter (GWRC) 37 that has a diameter of 1.33 millimeters and a length that is so the distal part 41 of the GWRC 37 engages the intermediate port 32 of the IC 20 when the proximal part 39 of the GWRC 37 is positioned at the proximal port 30 of the IC 20.

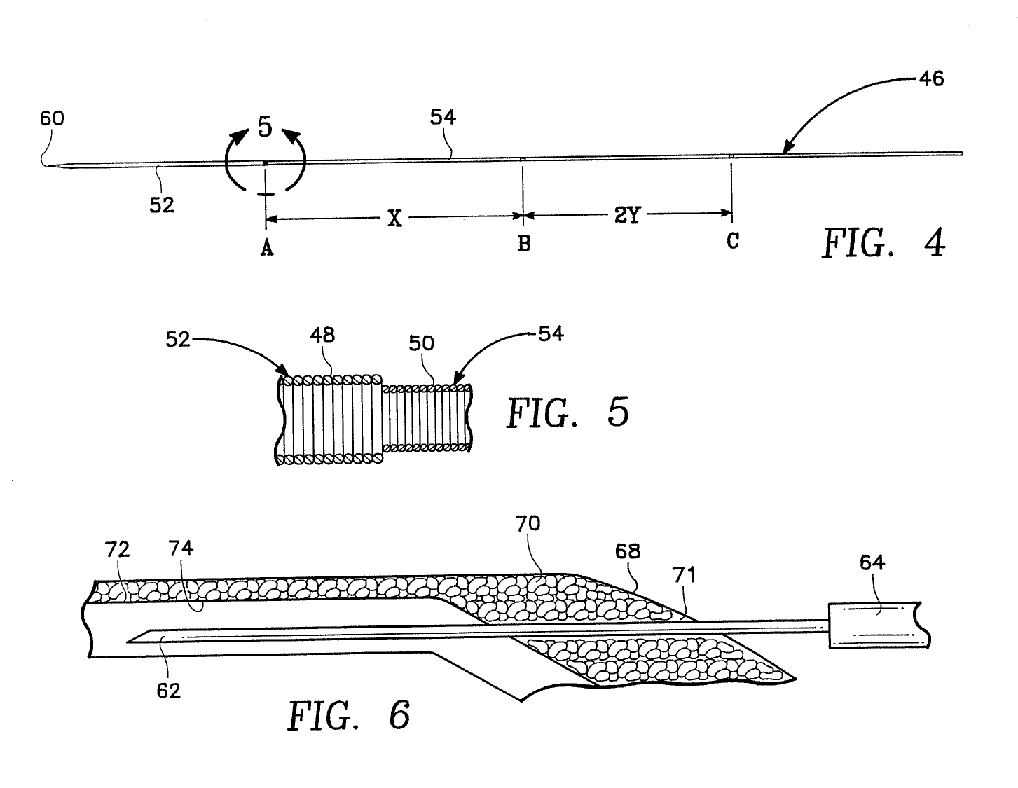

Referring particularly to FIGS. 4 and 5, there is shown the sharp tipped guide wire (STGW) 46. Indicating mark “A” on the STGW 46 denotes the junction between a stiffer distal part 52 and a less stiff proximal part 54. When indicating mark “B” on the STGW is aligned with the proximal part 39 of the GWRC 37, mark “A” will be at the hook shaped distal end 41 of the GWRC 37. When indicating mark “C” on the STGW 46 is aligned with the proximal port 30 of the IC 20, a portion of the proximal part 54 of the STGW 46 is prolapsed into the intermediate section 23 of the IC 20. The length of the prolapse is “Y” so the distance between indicating marks “B” and “C” will be 2Y since the prolapse constitutes a doubled up section of the STGW, in other words, folded upon itself.

The typical construction of STGW 46 would be a wire that is formed of a tightly packed series of coils 48 and a tightly packed series of coils 50. In actual practice, the diameter of the STGW 46 will generally be no more than approximately 0.035 inches. The coils 48 are of a slightly greater diameter than coils 50. Coils 48 are located at the distal part 52. The coils 50 are located within the proximal part 54 of the STGW 46 which is the remainder of the STGW 46 which is not distal part 52. The length of the portion of the proximal part 54 between the mark “B” and the mark “A” is denoted as distance “X” in FIG. 4. Because of the slightly less overall diameter of the coils 50, the proximal part 54 is substantially less stiff than the distal part 52. However, the distal part 52 can readily bend. The distal part 52 ends at a sharpened point 60. The function of the sharpened point 60 will be explained further on in the specification. It is to be noted that the STGW 46 could be constructed in numerous different manners so as to form a highly flexible proximal part 54. Also, the STGW 46 could be constructed other than metal such as plastic which does not include coils. Also, the diameter of the coils of the proximal part 54 could be of the same diameter as the distal part 52 but where the coils 50 within the proximal part 54 are just made of weaker material.

Referring particularly to FIG. 6, a cannula such as an eighteen gauge thin walled needle 62, which is placed on a syringe 64, is used to penetrate the skin surface 68 of the skin 70 producing a first puncture site 71 and then penetrate the wall of the vein 72.

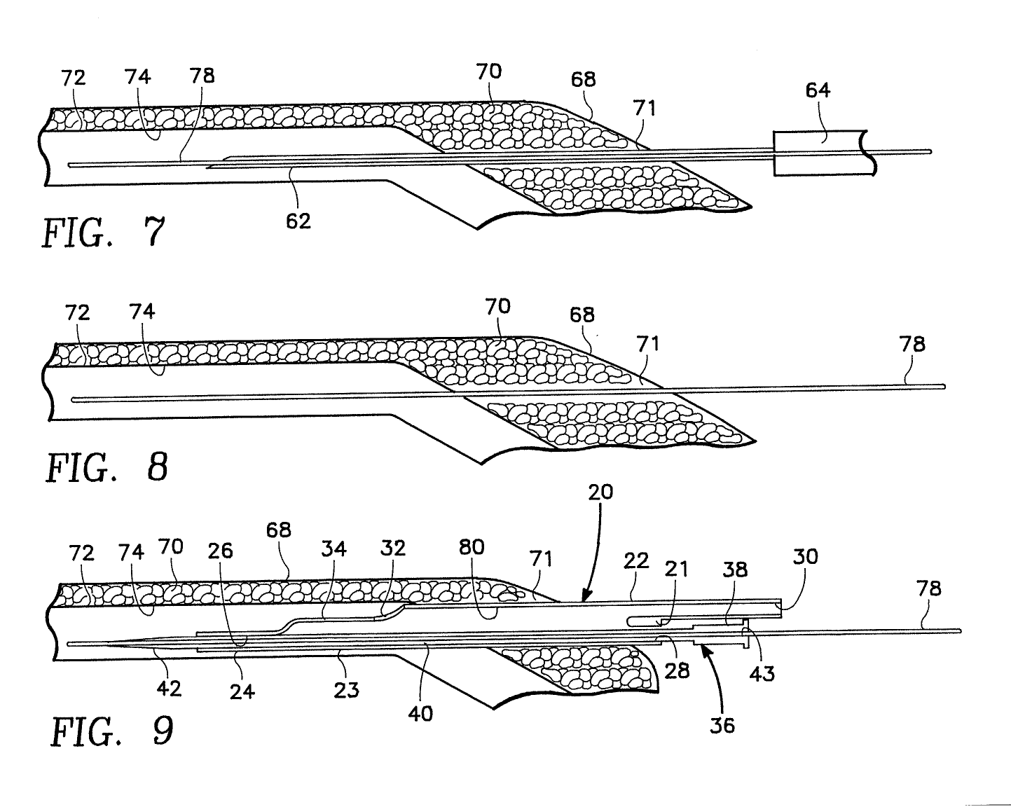

Referring particularly to FIG. 7, the syringe 64 is removed and first guide wire (GW) 78 is placed through needle 62 into the lumen 74 of the vein 72. The first GW is deemed to be conventional and has a constant diameter and a constant stiffness along its entire length.

Referring particularly to FIG. 8, the needle 62 is removed leaving only a first GW 78 with access into the lumen 74 of the vein 72. The insertion of this GW 78 within the vein 72 is accomplished by way of the standard modified Sellinger technique.

Referring particularly to FIG. 9, the DC 36 is inserted through port 28 of the IC 20 and both are advanced as a unit over the GW 78 into the lumen 74 of the vein 72. DC 36 establishes a close fit with the distal port 26 preventing blood from being aspirated from the distal port 26.

It is to be noted that when the intermediate port 32 is located within the lumen 74 of the vein 72, that blood can be aspirated from proximal port 30 of the IC 20 thereby confirming that the IC 20 is within the lumen 74. This aspirating position is shown in FIG. 9 of the drawings with it being understood that there would be a syringe (not shown) connecting with the proximal port 30 to affect the aspiration.

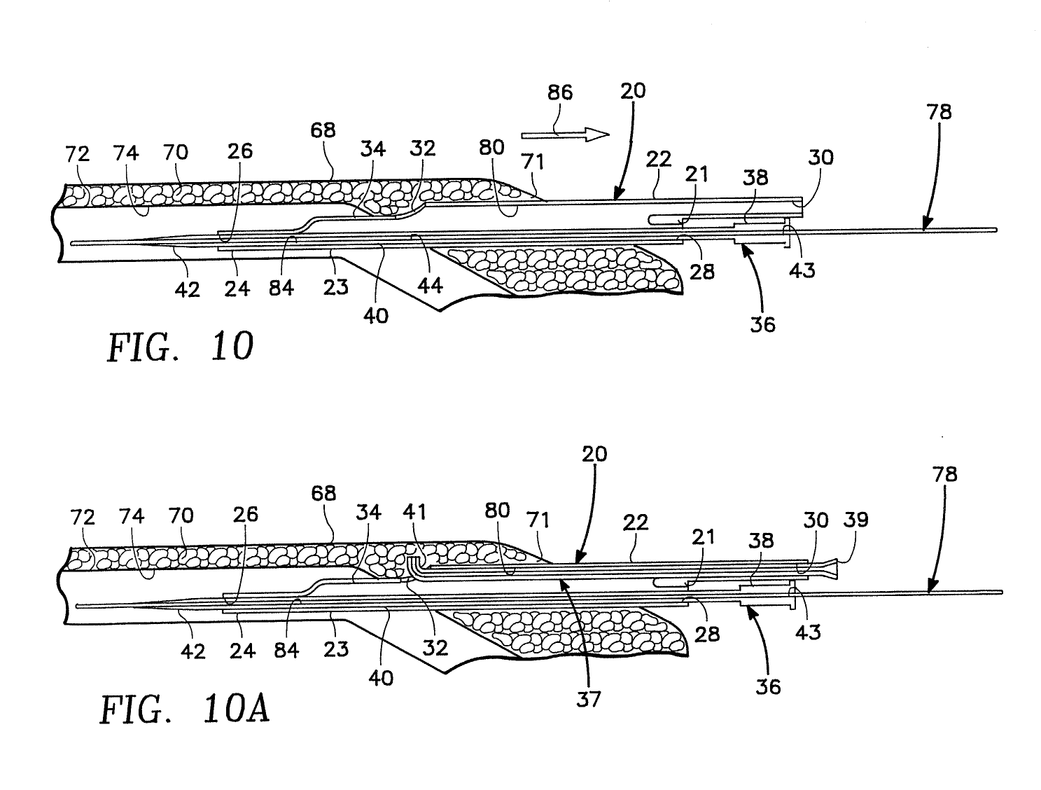

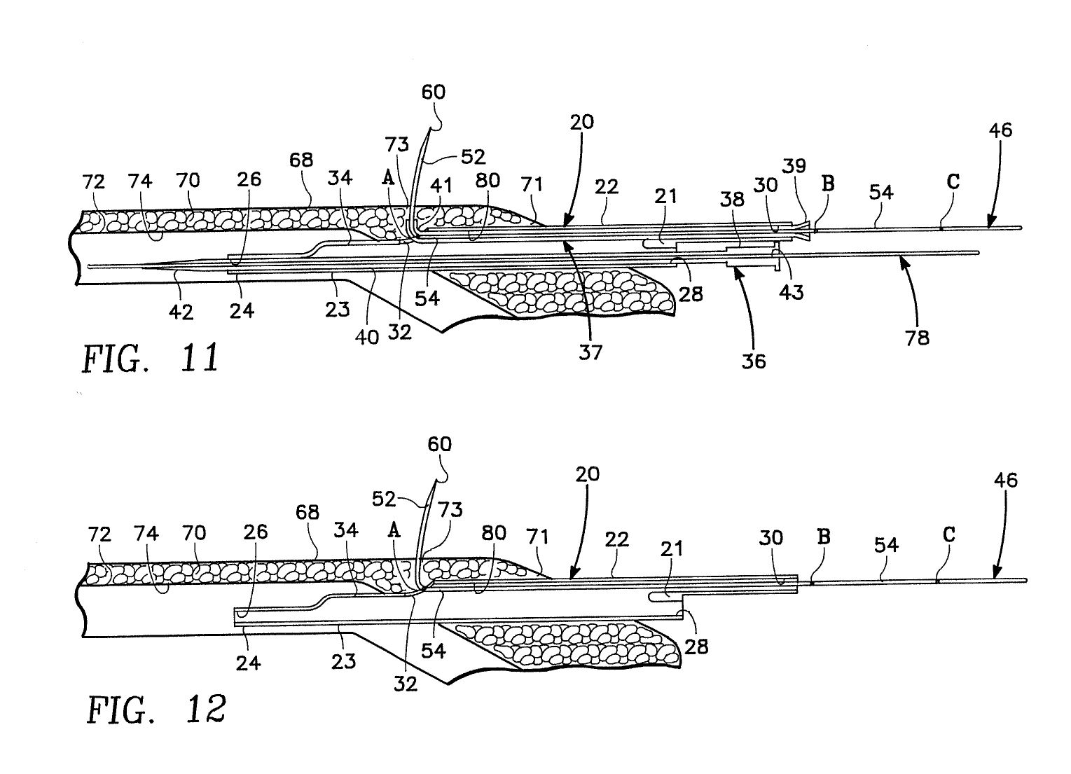

The IC 20 is then to be withdrawn slightly and a further attempt is made to aspirate blood. If blood can still be aspirated, the IC 20 is withdrawn again a slight amount. At some point, the installer will no longer be able to aspirate blood and the installer will then know that the intermediate port 32 is located just exteriorly of the vein 72 with such being clearly shown in FIG. 10 of the drawings. The movement of the IC 20 in the withdrawing direction is indicated by arrow 86. The GWRC 37 is advanced through proximal port 30 until the distal end 41 engages with the intermediate port 32. The installer has made sure that a plane connecting proximal ports 28 and 30 is aligned parallel to the surface 68 of the skin 70. This insures that the intermediate port 32 will be positioned in a direction facing parallel to the surface 68 of the skin 70. The installer then advances the STGW 46 through the GWRC 37 until the sharpened point 60 will exit the distal end 41 of GWRC 37 which has engaged the intermediate port 32. The hook shaped distal end 41 redirects the STGW 46 subcutaneously and parallel to the surface of the skin 68 such that the STGW 46 courses subcutaneously some distance and is then manipulated to puncture the skin 68 from the inside producing a second puncture site 73. The result is that the sharpened point 60 will penetrate the skin 70 and protrude exteriorly of the skin surface 68 which is some distance from the first puncture site 71 where the vein is punctured. This position is clearly shown in FIG. 11 of the drawings. In referring to FIGS. 11 and 12, the distance from intermediate port 32 to where the skin is punctured by the sharpened point 60 is actually several inches although in FIGS. 11 and 12 it appears to be a relatively short distance.

The installer is to continue moving the STGW 46 through the GWRC 37 until calibration mark “B” is located directly adjacent the proximal port 39 of the GWRC 37. This position is shown in FIG. 11. This will inform the installer that the transition point between the distal part 52 and the proximal part 54 (indicating mark A) is located directly adjacent the intermediate port 32.

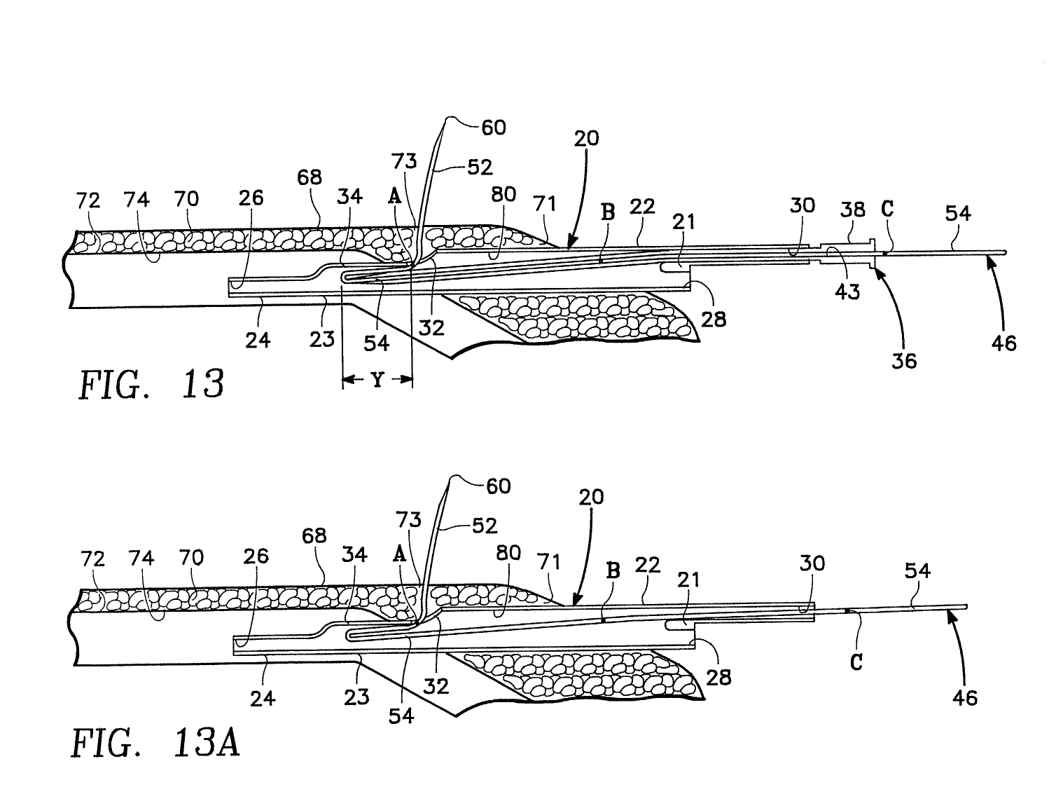

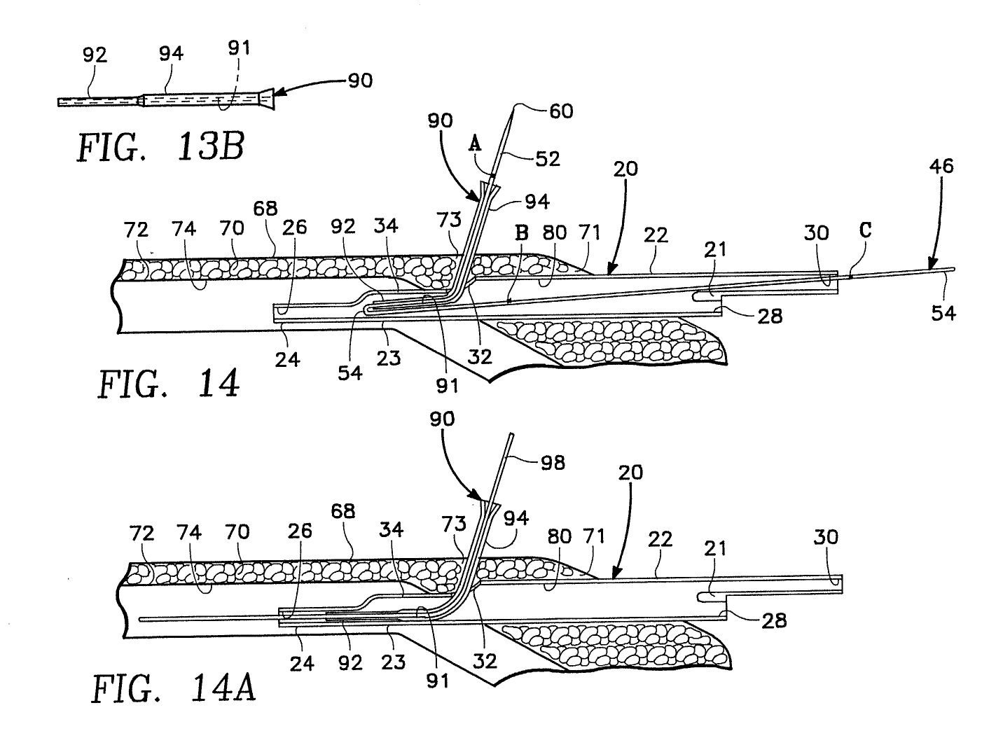

At this time, the GW 78 and the DC 36 are withdrawn from proximal port 28. GWRC 37 is withdrawn from proximal port 30. This position is shown in FIG. 12 of the drawings. DC 36 is then reused by being placed over the proximal part 54 of the STGW 46. The DC 36 is then advanced to a totally inserted position within the IC 20 to cause the proximal part 54 to prolapse (double on itself) into the intermediate section 23 of the IC 20. This prolapsing is facilitated by the weak coils of proximal part 54. This prolapsing must occur within the length “X” with the overall distance of the prolapse being “Y” therefore utilizing a length of 2Y of the length “X”. The calibration mark “B” insures that the stiffer distal part 52 is not subject to prolapsing since it is located in alignment with the intermediate port 32. This position is clearly shown in FIG. 13 of the drawings. At this time, DC 36 can be withdrawn, as shown in FIG. 13A.

It is possible that instead of prolapsing the STGW 46 there may be used a snare (not shown) to manipulate the proximal part 54 of STGW 46 with the snare being attached to the end of proximal part 54 and then be advanced to the intermediate port 32 by withdrawing of the STGW 46 from the intermediate port 32 pulling the STGW 46 away from the surface of the skin 68. At this time, the snare is advanced into the intermediate section 23 of the IC 20 carrying with it the proximal part 54 of the STGW 46 in a retracted direction (reverse to the withdrawal direction). The snare is then released and removed leaving the proximal part 54 coursing directly from the intermediate port 32 into the distal portion of the IC 20 comprised of sections 23 and 24.

Referring specifically to FIGS. 13B and 14, a second DC 90 is then advanced over the distal part 52 and the sharpened point 60 of the STGW 46, through the intermediate port 32 and into the intermediate portion 23. It is to be noted that the second DC 90 has a distal part 92 which is 1.33 millimeters in diameter and which has a through hole 91 which is slightly larger so as to allow for the passage of the STGW 46, and a proximal part 94 which is 2.67 millimeters in diameter. At this time, the STGW 46 is to be withdrawn from the second DC 90. An extra support guide wire (ESGW) 98 is then passed through the second DC 90 and advanced within the lumen 74 of the vein 72 (FIG. 14A). The wider proximal part 94 is forced into the intermediate portion 23 and the distal portion 24 of the IC 20. Because the diameter of the proximal part 94 is greater than the internal diameter of the common lumen 80 within the distal portion 24, the frangible seam 34 is caused to break. Because the frangible seam 34 extends from intermediate port 32 to distal end 26, it allows for removal of introducer catheter 20 while maintaining position of extra support guide wire 98 which punctures the skin at the second puncture site 73 which is different and spaced from first puncture site 71.

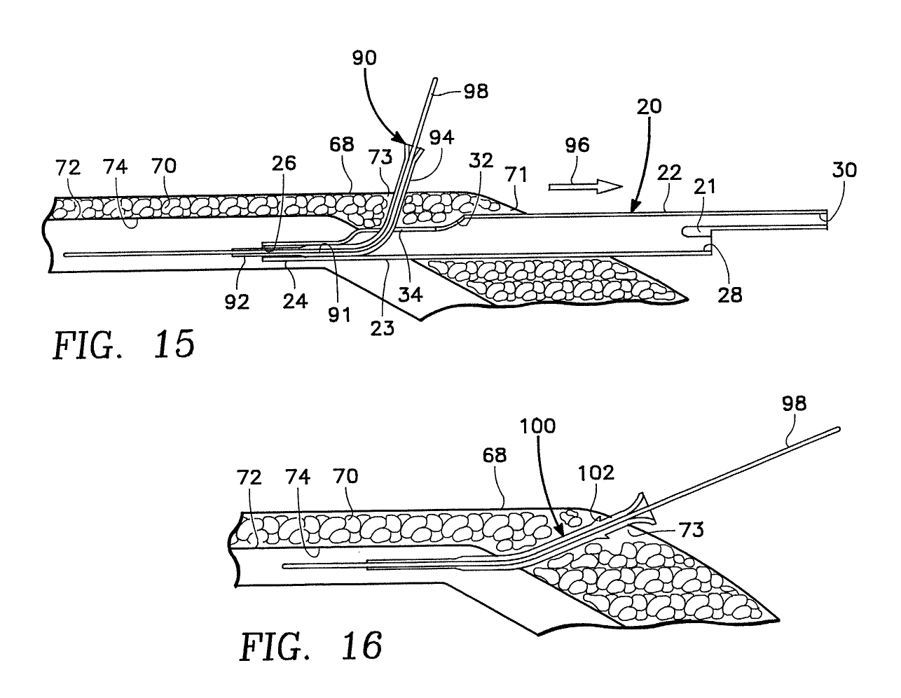

At this time, the IC 20 is withdrawn as indicated in the direction of arrow 96 in FIG. 15 with the result that the frangible seam 34 becomes totally broken permitting the withdrawal of the IC 20 and leaving in place the distal part 92 and a portion of proximal part 94 of the second DC 90 installed within the lumen 74 of the vein 72. The second DC 90 is then removed leaving only the ESGW 98 installed within the lumen 74 of the vein 72. It is to be noted that the entry point of the ESGW 98 is spaced several inches subcutaneously from the vein 72.

A STIC can be advanced over the ESGW 98 which can be used to introduce temporary transvenous pacing wires, a Swan-Ganz catheter, a triple lumen catheter or a hemodialysis access catheter with a typical such catheter 100 being shown in FIG. 16. The catheter 100 would then be sutured in place. It is recommended that the exterior surface of the catheter 100 be impregnated with an antibiotic to further reduce the possibility of infection. Catheter 100 could also be modified to have at its proximal end an annular barb 102. The purpose of the barb 102 is to help secure the catheter 100 within the skin 90 to hopefully eliminate slippage of the catheter 100 outwardly from the puncture side of the skin 68. Such slippage and subsequent readvancement of the catheter 100 can contribute significantly to infection.

What is claimed is:

1. A non-surgical placement of a subcutaneously tunneled central venous semipermanent catheter through the skin and into a vein comprising the steps of:
inserting a needle into a central vein with the proximal end of the needle protruding exteriorly of the vein;
inserting a first guide wire through the needle and into the vein;
removing of the needle;
utilizing an introducer catheter that has a common lumen extending between a pair of proximal ports located at a proximal part and a distal port located at a distal part with said proximal part being defined as the portion of said introducer catheter between said proximal ports and an intermediate port and said distal part being defined as the portion of said introducer catheter between the intermediate port and said distal port;
inserting said introducer catheter onto said first guide wire with said first guide wire passing entirely through said introducer catheter;
installing a first dilator catheter onto said first guide wire and inserting said first dilator catheter through one of said proximal ports and into said common lumen;
positioning said distal part of said introducer catheter within the vein and the proximal part of said introducer catheter located exteriorly of the skin wherein said intermediate port is located between said proximal ports and said distal port and a frangible longitudinal seam extending between said intermediate port and said distal port;
positioning said intermediate port directly adjacent the vein and exteriorly of the vein but still beneath the skin;
inserting a redirecting catheter into said introducer catheter with a distal part of said redirecting catheter engaging with said intermediate port;
utilizing a second guide wire that is flexible and basically straight with a sharp distal tip and a stiff distal portion and a more flexible proximal portion;
inserting said second guide wire through said redirecting catheter to exit said intermediate port of said introducer catheter and travel subcutaneously some distance before said sharp distal tip exits the surface of the skin;
removing said dilator catheter, said first guide wire and said redirecting catheter from said introducer catheter;
causing said second guide wire to prolapse and extend into said distal part of said introducer catheter;
inserting a second dilator catheter over said stiff distal portion of said second guide wire and passing said second dilator catheter through said intermediate port into said distal part of said introducer catheter engaging with said prolapse;
removing said second guide wire from said introducer catheter;
inserting an extra support guide wire within said second dilator catheter and into said distal part;
removing of said introducer catheter by having said second dilator catheter pass through said frangible longitudinal seam; and
removing said second dilator catheter leaving said extra support guide wire in place which is adapted to be used to gain access into said vein by a semipermanent catheter.

2. The method as defined in claim 1 wherein the fifth inserting step includes:
utilizing an indicating mark on said second guide wire that when aligned with said proximal ports indicates that said second guide wire is fully inserted within said introducer catheter.

3. The method as defined in claim 1 wherein said causing step comprises:
utilizing a further dilator catheter to be inserted over said proximal portion of said second guide wire and inserting same far enough to cause said prolapse.

4. A catheter assembly to be installed through skin and into a vein comprising:
an introducer catheter which has a single common lumen extending between a pair of side-by-side proximal ports and a distal port, whereby said distal port is to be located within the vein and the proximal ports to be located exteriorly of the skin.

5. The catheter assembly as defined in claim 4 wherein:
said introducer catheter has an intermediate port located between said proximal ports and said distal port, said intermediate port is to be used for passage of a prolapsible guide wire which is to tunnel subcutaneously before penetrating the skin where said guide wire is to double over upon itself when prolapsing.

6. The catheter assembly as defined in claim 5 wherein:
said introducer catheter having a longitudinal frangible seam located within a distal portion of said introducer catheter defined as extending between said intermediate port and said distal port, said frangible seam to be broken to permit withdrawal of said introducer catheter during the installing of said catheter assembly.

7. The catheter assembly as defined in claim 6 wherein:
said distal portion including a small diameter portion and a larger diameter portion with prolapsing of said guide wire to be doubled over on itself to occur within said larger diameter portion when inserted within said introducer catheter.

8. A catheter assembly to be installed through skin and into a vein comprising:
a guide wire having a thin elongated body, said elongated body having a distal section which is bendable, said elongated body having a proximal section connecting with said distal section, said proximal section being substantially of less thickness than said distal section thereby being weaker permitting said proximal section to be bent over upon itself permitting prolapse of a portion of said proximal section of said body within a catheter.

9. The catheter assembly as defined in claim 8 wherein:
said elongated body being constructed of wire that is formed into tightly wound coils.

10. The catheter assembly as defined in claim 8 wherein:
said distal section terminating in a sharpened point adapted for penetrating the skin.

11. The catheter assembly as defined in claim 8 comprising:
said catheter being entirely hollow defining a common lumen between a pair of proximal ports and a distal port, whereby said distal port is to be located within a vein and said proximal ports are to be located exteriorly of the skin.

12. The catheter assembly as defined in claim 11 wherein said catheter is further defined as having:
an intermediate port located between said proximal ports and said distal port, whereby said intermediate port is to be used for passage of a guide wire which is to penetrate the skin.

13. The catheter assembly as defined in claim 12 wherein said catheter is further defined as having:
a longitudinal frangible seam located between said intermediate port and said distal port, whereby said frangible seam is to be broken to permit withdrawal of said catheter during a vein installing procedure.

14. The catheter assembly as defined in claim 13 wherein:
said longitudinal frangible seam being formed within a portion of said catheter that has a smaller diameter section and a larger diameter section with said larger diameter section to accommodate said prolapse of said proximal part of said guide wire.

15. A catheter assembly to be installed through skin and into a vein comprising:
an introducer catheter which has a single common lumen extending between a pair of proximal ports located in juxtaposition and a distal port, whereby said distal port is to be located within the vein and said proximal ports are to be located exteriorly of the skin; and
a guide wire to be inserted within said introducer catheter, said guide wire to be prolapsible by being able to be bent over itself within said lumen, a dilator catheter to be installed in conjunction with said guide wire.

16. The catheter assembly as defined in claim 14 wherein:
said introducer catheter has an intermediate port located between said proximal ports and said distal port, said guide wire to be connected through said intermediate port.

17. The catheter assembly as defined in claim 15 wherein:
said introducer catheter having a longitudinal frangible seam located between said distal port and said internal port, said frangible seam to be broken to permit withdrawal of said introducer catheter leaving in place said guide wire.

18. The catheter assembly as defined in claim 15 wherein:
said guide wire having a thin elongated body, said elongated body having a distal section, said elongated body having a proximal section connecting with said distal section, said proximal section being substantially of less thickness than said distal section.

19. The catheter assembly as defined in claim 15 wherein:
said elongated body being constructed of wire that is formed into tightly wound coils.

20. The catheter assembly as defined in claim 15 wherein:
said distal section terminating in a sharpened point adapted for penetrating the skin.

21. The catheter assembly as defined in claim 15 wherein:
said common lumen being entirely hollow between said proximal ports and said distal port.
